(19) United States
(12) Patent Application Publication
(10) Pub. No.: US 2009/0317842 A1
(43) Pub. Date: Dec. 24, 2009

(54) Methods and Tools for The Therapy of Neurodegenerative Pathologies

(75) Inventors: Fabien Schweighoffer, Nogent Sur Marne (FR); Laurent Desire, Paris (FR); Jérôme Bourdin, Grenoble (FR)

Correspondence Address: NIXON & VANDERHYE, PC, 901 NORTH GLEBE ROAD, 11TH FLOOR, ARLINGTON, VA, 22203, US

(73) Assignee: EXONHIT THERAPEUTICS SA, Paris (FR)

(21) Appl. No.: 12/309,492

(22) PCT Filed: Jul. 20, 2007
(86) PCT No.: PCT/FR2007/051706

§ 371 Date: May 6, 2009

(30) Foreign Application Priority Data
Jul. 21, 2006 (FR) 0606698

(51) Int. Cl. G01N 33/53; C07D 471/04; C07D 243/14

(52) U.S. Cl. 435/7.92; 435/7.1; 546/120; 540/569

(57) ABSTRACT

The present invention concerns compositions and methods for the treatment of neurodegenerative diseases in which the cognitive functions are altered, such as observed in Alzheimer's disease. More particularly, the invention presents a strategy for human clinical monitoring of the activity and/or effectiveness of neuroprotective treatments, based on biochemical assay of certain platelet parameters, and thus can be done by blood sampling. The invention also concerns methods, tools, constructions and compositions suitable for implementing these strategies.

The present invention concerns compositions and methods for the treatment of neurodegenerative diseases in which the cognitive functions are altered, such as observed in Alzheimer's disease. More particularly, the invention presents a strategy for human clinical monitoring of the activity and/or effectiveness of neuroprotective treatments, based on biochemical assay of certain platelet parameters, and which therefore can be done by blood sampling. The invention also concerns methods, tools, constructs and compositions suitable for implementing these strategies.

Alzheimer's disease represents the principal cause of dementia and the most common neurodegenerative disease. This progressive disease is characterized by memory loss and by degradation of language ability, orientation and judgment. Examination of the brain of patients suffering from this disease shows a loss of neurons of the hippocampus, an important memory center, and of the cerebral cortex, involved in reasoning, language and memory. Cholinergic neurons are particularly affected by this depletion.

A major anomaly observed in the brains of patients suffering from Alzheimer's disease is the accumulation of intracellular and extracellular protein aggregates. Senile plaques formed by intra- and extracellular aggregation of amyloid beta peptide (Aβ), resulting from cleavage of APP (Amyloid Precursor Protein), characterize regions of alterations involving neurons and glial cells. Other intracellular aggregates, neurofibrillary tangles, and tau protein, seem to correlate closely with the seriousness of the dementia.

Genetic studies conducted on familial forms have shown that 4 genes are associated with developing the disease. APP, presenilins 1 and 2 (PS1 and PS2) and apolipoprotein E (Apo E), Although mutations or polymorphisms in each of these genes lead to an increased production of Aβ peptide, the mechanisms that preside in synaptic and neuronal losses remain poorly understood. Several hypotheses and mechanisms therefore seem to coexist, involving phenomena such as oxidative stress, which may notably be induced by Aβ peptide, inflammatory and immune phenomena, or even sex hormone deficiencies, insulin deficiencies and hypothyroidism. Other hypotheses emphasize the role of changes in calcium influx and excitotoxicity. However, no element has permitted completely accounting for the particular vulnerability of cholinergic neurons. The treatments currently available, based on the use of acetylcholinesterase inhibitors, only temporarily improve patients' cognitive functions and do not represent a therapeutic approach that can slow the progression of Alzheimer's disease, still less combat it.

Although recent observations emphasize the possibility of intervening pharmacologically by immunotherapy approaches directed against Aβ peptide, directly targeting secretases, which are proteases involved in APP metabolism and Aβ peptide production is even more pertinent.

Aβ peptide is a fragment of 40/42 residues that is produced, in the amyloidogenic pathway, via sequential cleavage of the APP protein by two proteases called β-secretase (BACE) and γ-secretase (presenilins). The sequence for the Aβ peptide is located at the junction between the intramembrane and extracellular domains of APP. In the non-amyloidogenic pathway, APP is cleaved in the Aβ domain by an α-secretase between amino acids 16(Lys) and 17(Leu) of the Aβ region, generating the soluble APP α portion (sAPPα, 105-125 kDa, residues 1-688 of the APP770 form) salted out in the extracellular medium and a fragment retained at the membrane (containing a part of the transmembrane domain and the C-terminal intracellular part) called C83 (10 kDa), itself cleaved by γ-secretase to generate the “APP IntraCellular Domain” peptide (AICD) and P3 peptide (3 kDa). The action of α-secretase therefore not only impedes the formation of the amyloid peptide, but also stimulates the generation of the large extracellular N-terminal fragment (ectodomain) of APP. The soluble N-terminal fragments of APP generated by α-secretase, or sAPPα, are salted out constitutively in the vesicular lumen and at the surface of the cell. Such species of APP are secreted, in vitro, in culture medium conditioned by cells expressing APP, and are found in vivo in the plasma and cerebrospinal fluid.

The approaches described to stimulate the activity of α-secretase and increase the levels of sAPPα, involve the activation of G-protein-coupled receptors such as the P2Y2 nucleotide receptors, the PACAP PAC1 receptor, or receptors for various neurotransmitters such as muscarinic receptors, the metabotropic glutamate receptor or even serotonin receptors (refer to section iii for a review). The pathways stimulated by neurotransmitters involve the protein kinase C (PKC) and phospholipase C systems, as well as the MAP kinases, well described in the literature as sAPPα production modulators, such as the stimulation of PKC-dependent pathways by phorbol esters or even by serotonin 5-HT2a and 2c receptor agonists. Other pathways involve the serotonin 5-HT(4) receptor, known to play a role in cognition and memory, via the production of cAMP and the recruitment of Rac1 GTPase or even acetylcholine inhibitors via PKC and/or MAP kinases, estrogens such as 17β-estradiol or even testosterone. Certain hormones and growth factors such as EGF and insulin are generally known for stimulating the production of sAPPα via PKC or P13K, respectively. Other pharmacological agents have been recently described as stimulating the production of sAPPα, according to a cAMP-protein kinase A (PKA) pathway, such as forskolin, or according to a PKC/MAP-kinase pathway, such as nonsteroidal antiinflammatories like cyclooxygenase COX inhibitors (ibuprofen), statins inhibitors of HMG-CoA reductase (lovastatin), rasagiline derivatives or even polyphenols like (−)-epigallocatechin-3-gallate. But all these approaches, while they permit validating the pertinence of the strategy seeking to stimulate sAPPalpha production by means of pharmacological tools, have not led to new compounds suitable for human clinical use.

The present invention provides a rationale for the use of pharmacological agents such as chemical compounds belonging to the pyrazolopyridine class, including etazolate, intended to stimulate the production of the sAPPα fragment.

The present invention also describes the link between increased production of sAPPalpha and the capacity of etazolate to inhibit the effects induced by ROS (“Reactive Oxygen Species”), i.e., oxidative stress.

This oxidative stress phenomenon plays an essential role in several aspects of Alzheimer's disease: not only neuronal degeneration and astrocyte inflammation, but also platelet activation and aggregation. These latter phenomena participate in the vascular complications of Alzheimer's disease and are the cause of vascular dementia.

Consequently, it is possible to monitor an etazolate inhibitor effect on pathways initiated by oxidative stress at the level of platelet activation. More generally, it is possible to monitor the inhibitor effect of neuroprotective compounds at the level of platelet activation.

Thus, for the first time, the present invention permits proposing the measurement of any biological phenomenon linked to platelet activation or aggregation for clinical or therapeutic monitoring of the effectiveness of neuroprotective compounds. The capacity of generating Abeta and sAPPalpha peptides from APP is shared by the nervous system and the platelets. Consequently, since the inhibitory action of etazolate on oxidative stress is translated by an increase of sAPPalpha production, the present invention provides a rational for monitoring the action of etazolate on APP maturation from platelet samples or from blood samples more generally.

The present invention also claims the measurement of any biological phenomenon linked to platelet activation or aggregation for clinical or therapeutic monitoring of the effectiveness of any compound of the pyrazolopyridine family. Moreover, the present invention permits claiming the measurement of any change in APP maturation in the blood, notably the concentration of sAPPalpha, from blood samples or platelet preparations, in order to ensure clinical and therapeutic monitoring of the effectiveness of any compound of the pyrazolopyridine family.

Thus, an object matter of the invention resides in a method to evaluate or monitor the effectiveness of a neuroprotective treatment in mammals, comprising a step of measuring (preferably in vitro or ex vivo) the production of sAPPalpha in a biological sample from the mammal having received said treatment, said sample containing platelets, the production of sAPPalpha being an indication of treatment effectiveness.

Another object of the invention resides in a process for immunological dosing of sAPPalpha in a sample, comprising a step of thermal treatment of the sample (to unmask the sAPPalpha), and a step of immunological dosing. The process is suited for dosing sAPPalpha from any sample, and notably blood or blood-derivative samples (serum, platelets, etc), or other biological fluids. The sample may be pretreated, notably by dilution, enrichment, filtration, etc.

### Neuroprotective Treatment

The invention may also be used to evaluate or monitor the effectiveness of any neuroprotective treatment in mammals. In the sense of the invention, by neuroprotective treatment is meant any treatment usable or used in the treatment of diseases affecting the nervous system, notably neurodegenerative diseases. In this context, compounds chosen from among pyrazolopyridines and GABA(A) receptor modulators can be named.

In the sense of the invention, a compound of the pyrazolopyridine family advantageously designates any compound of formula (I) below, substituted or unsubstituted at any position

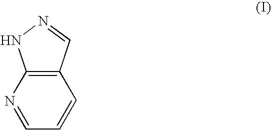

Compounds of the pyrazolopyridine family used in the present invention are, in particular, chosen from among the following compounds:
Etazolate of formula (II) below:

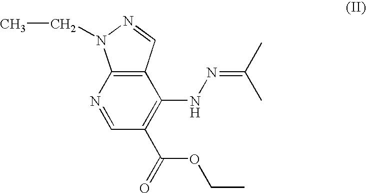

Etazolate constitutes a preferred embodiment of the invention.
4-butylamino-1-ethyl-6-methyl-1H-pyrazolo[3,4-b]pyridine-5-carboxylic acid ethyl ester (tracazolate),
4-butylamino-1-ethyl-1H-pyrazolo[3,4-b]pyridine-5-carboxylic acid ethyl ester,
1-(4-amino-pyrazolo[3,4-b]pyridin-1-yl)-β-D-1-deoxy-ribofuranose
1-ethyl-4-(N′-isopropylidene-hydrazino)-1H-pyrazolo[3,4-b]pyridine-5-carboxylic acid ethyl ester (SQ 2009)
4-amino-6-methyl-1-n-pentyl-1H-pyrazolo[3,4-b]pyridine
4-Amino-1-ethyl-6-methyl-1H-pyrazolo[3,4-b]pyridine-5-carboxylic acid ethyl ester (desbutyl tracacolate)
4-amino-1-pentyl-1H-pyrazolo[3,4-b]pyridine-5-carboxamide,
1-ethyl-6-methy 1-4-methylamino-1H-pyrazolo[3,4-b]pyridine-5-carboxylic acid ethyl ester,
4-amino-6-methyl-1-propyl-1H-pyrazolo[3,4-b]pyridine-5-carboxylic acid ethyl ester,
1-ethyl-4-ethylamino-6-methyl-1H-pyrazolo[3,4-b]pyridine-5-carboxylic acid ethyl ester,
4-amino-1-butyl-6-methyl-1H-pyrazolo[3,4-b]pyridine-5-carboxylic acid ethyl ester
5-(4-amino-pyrazolo[3,4-b]pyridin-1-yl)-2-hydroxymethyl-tetrahydrofuran-3-ol,
1-allyl-4-amino-6-methyl-1H-pyrazolo[3,4-b]pyridine-5-carboxylic acid allyl ester,
4-amino-6-methyl-1-pentyl-1H-pyrazolo[3,4-b]pyridine-5-carboxylic acid,
4-amino-1-ethyl-3,6-dimethyl-1H-pyrazolo[3,4-b]pyridine-5-carboxylic acid ethyl ester,
4-dimethylamino-1-ethyl-6-methyl-1H-pyrazolo[3,4-b]pyridine-5-carboxylic acid ethyl ester,
1-ethyl-6-methyl-4-propylamino-1H-pyrazolo[3,4-b]pyridine-5-carboxylic acid ethyl ester,
4-amino-1-pentyl-1H-pyrazolo[3,4-b]pyridine-5-carboxylic acid ethyl ester,
4-amino-6-methyl-1-pent-4-ynyl-1H-pyrazolo[3,4-b]pyridine-5-carboxylic acid ethyl ester,
4-amino-1-but-3-enyl-1H-pyrazolo[3,4-b]pyridine-5-allylamide,
4-amino-1-pentyl-1H-pyrazolo[3,4-b]pyridine-5-isopropylamide,
4-amino-1-pentyl-N-n-propyl-1H-pyrazolo-[3,4-b]pyridine-5-carboxamide,
4-amino-1-butyl-6-methyl-1H-pyrazolo[3,4-b]pyridine-5-carboxylic acid allyl ester,
4-amino-6-methyl-1-pent-3-ynyl-1H-pyrazolo[3,4-b]pyridine-5-carboxylic acid ethyl ester,
4-amino-1-pentyl-1H-pyrazolo[3,4-b]pyridine-5-prop-2-ynylamide
4-amino-1-(3-methyl-butyl)-1H-pyrazolo[3,4-b]pyridine-5-carboxylic acid allyl ester,
4-amino-1-pentyl-1H-pyrazolo[3,4-b]pyridine-5-N-(2-propenyl)carboxamide,
4-amino-1-pentyl-1H-pyrazolo[3,4-b]pyridine-5-carboxylic acid allyl ester,
4-amino-1-pentyl-1H-pyrazolo[3,4-b]pyridine-5-butylamide,
4-amino-1-but-3-ynyl-6-methyl-1H-pyrazolo[3,4-b]pyridine-5-carboxylic acid allyl ester,
4-amino-1-but-3-enyl-6-methyl-1H-pyrazolo[3,4-b]pyridine-5-carboxylic acid allyl ester,
4-amino-6-methyl-1-pentyl-1H-pyrazolo[3,4-b]pyridine-5-allylamide,
4-amino-6-methyl-1-pentyl-1H-pyrazolo[3,4-b]pyridine-5-carboxylic acid allyl ester,
4-amino-6-methyl-1-(3-methyl-butyl)-1H-pyrazolo[3,4-b]pyridine-5-carboxylic acid allyl ester,
4-amino-6-methyl-1-pentyl-1H-pyrazolo[3,4-b]pyridine-5-carboxylic acid isobutyl ester,
4-amino-6-methyl-1-pentyl-1H-pyrazolo[3,4-b]pyridine-5-butylamide,
4-amino-6-methyl-1-(3-methyl-but-2-enyl)-1H-pyrazolo[3,4-b]pyridine-5-carboxylic acid allyl ester,
4-amino-1-pentyl-1H-pyrazolo[3,4-b]pyridine-5-cyclopropylamide,
ethyl-4-amino-1-pentyl-1H-pyrazolo[3,4-b]pyridine-5-hydroxamate,
4-amino-6-methyl-1-pentyl-1H-pyrazolo[3,4-b]pyridine-5-carboxylic acid prop-2-ynyl ester,
4-amino-6-methyl-1-pent-4-ynyl-1H-pyrazolo[3,4-b]pyridine-5-carboxylic acid allyl ester,
4-amino-6-methyl-1-pent-4-enyl-1H-pyrazolo[3,4-b]pyridine-5-carboxylic acid allyl ester,
4-amino-1-pent-3-ynyl-1H-pyrazolo[3,4-b]pyridine-5-propylamide,
4-amino-1-pentyl-1H-pyrazolo[3,4-b]pyridine-5-cyclopropylmethyl-amide,
4-amino-6-methyl-1-pentyl-1H-pyrazolo[3,4-b]pyridine-5-carboxylic acid 2-methylallyl ester,
4-Amino-1-pent-3-ynyl-1H-pyrazolo[3,4-b]pyridines-5-allylamide (IC1 190,622),
4-amino-1-pent-4-ynyl-N-2-propenyl-1H-pyrazolo[3,4-b]pyridine-5-carboxamide,
4-amino-1-pent-3-ynyl-1H-pyrazolo[3,4-b]pyridine-5-prop-2-ynylamide,
4-amino-1-pentyl-1H-pyrazolo[3,4-b]pyridine-5-but-2-ynylamide,
4-amino-6-methyl-1-pent-3-ynyl-1H-pyrazolo[3,4-b]pyridine-5-carboxylic acid allyl ester,
4-amino-1-(2-cyclopropyl-ethyl)-6-methyl-1H-pyrazolo[3,4-b]pyridine-5-carboxylic acid allyl ester,
4-amino-1-hex-5-ynyl-6-methyl-1H-pyrazolo[3,4-b]pyridine-5-carboxylic acid allyl ester,
4-amino-1-pent-3-ynyl-1H-pyrazolo[3,4-b]pyridine-5-cyclopropylmethyl-amide,
4-amino-6-methyl-1-pentyl-1H-pyrazolo[3,4-b]pyridine-5-carboxylic acid but-3-enyl ester,
4-amino-6-methyl-1-pentyl-1H-pyrazolo[3,4-b]pyridine-5-carboxylic acid cyclopropylmethyl ester,
4-butylamino-1-pentyl-1H-pyrazolo[3,4-b]pyridine-5-allylamide,
4-amino-6-methyl-1-pentyl-1H-pyrazolo[2,4-b]pyridine-5-carboxylic acid 2-cyclopropylethyl ester,
4-amino-6-methyl-1-pent-3-ynyl-1H-pyrazolo[3,4-b]pyridine-5-carboxylic acid cyclopropylmethyl ester,
4-amino-6-methyl-1-pent-4-ynyl-1H-pyrazolo[3,4-b]pyridines-5-carboxylic acid cyclopropylmethyl ester,
4-amino-1-benzyl-6-methyl-1H-pyrazolo[3,4-b]pyridine-5-carboxylic acid ethyl ester,
4-amino-1-pentyl-1H-pyrazolo[3,4-b]pyridine-5-benzylamide,
4-amino-1-pentyl-1H-pyrazolo[3,4-h]pyridine-5-phenylamide,
4-amino-6-methyl-1-pentyl-1H-pyrazolo[3,4-b]pyridine-5-carboxylic acid benzyl ester,
4-Azido-1-β-D-ribofuranosylpyrazolo[3,4-b]pyridine,
1-pent-3-ynyl-N-2-propenyl-4-propionamido-1H-pyrazolo[3,4-b]pyridine-5-carboxamide,
2-(4-amino-pyrazolo[3,4-b]pyridin-1-yl)-5-hydroxymethyl-tetrahydro-furan-3,4-diol,
2-(6-methyl-1H-pyrazolo[3,4-b]pyridin-4-ylamino)-ethanol,
3-(6-methyl-1H-pyrazolo[3,4-b]pyridin-4-ylamino)-propan-1-ol,
3-(6-methyl-1H-pyrazolo[3,4-b]pyridin-4-ylamino)-acetic acid propyl ester,
2-(6-methyl-1H-pyrazolo[3,4-b]pyridin-4-ylamino)-propionic acid ethyl ester,
2-(6-methyl-1H-pyrazolo[3,4-b]pyridin-4-ylamino)-pentanoic acid ethyl ester,
2-(6-methyl-1H-pyrazolo[3,4-b]pyridin-4-ylamino)-benzoic acid ethyl ester,
3-(6-methyl-1H-pyrazolo[3,4-b]pyridin-4-ylamino)-pentanoic acid propyl ester,
N-benzylidene-N′-(3-methyl-1-phenyl-1H-pyrazolo[3,4-b]pyridin-4-yl)-hydrazine,
N-furan-2-ylmethylene-N′-(3-methyl-1-phenyl-1H-pyrazolo[3,4-b]pyridin-4-yl)-hydrazine,
N-(4-fluoro-benzylidene)-N′-(3-methyl-1-phenyl-1H-pyrazolo[3,4-b]pyridin-4-yl)-hydrazine,
N-(3-furan-2-yl-allylidene)-N′-(3-methyl-1-phenyl-1H-pyrazolo[3,4-b]pyridin-4-yl)-hydrazine,
N-(4-methoxy-benzylidene)-N-(3-methyl-1-phenyl-1H-pyrazolo[3,4-b]pyridin-4-yl)-hydrazine,
4-[(3-methyl-1-phenyl-1H-pyrazolo[3,4-b]pyridin-4-yl)-hydrazonomethyl]-benzonitrile,
N-benzo[1,3]dioxol-5-ylmethylene-N′-(3-methyl-1-phenyl-1H-pyrazolo[3,4-b]pyridin-4-yl)-hydrazine,
N-(3-methyl-1-phenyl-1H-pyrazolo[3,4-b]pyridin-4-yl)-N′-(4-nitro-benzylidene)-hydrazine,
N-(3-methy-1-phenyl-1H-pyrazolo[3,4-b]pyridin-4-yl)-N-(2-nitro-benzylidene)-hydrazine,
N-(3-methyl-1-phenyl-1H-pyrazolo[3,4-b]pyridin-4-yl)-N′-(4-trifluoromethyl-benzylidene)-hydrazine,
N-(3-methyl-1-phenyl-1H-pyrazolo[3,4-b]pyridin-4-yl)-N′-(5-nitro-furan-2-ylmethylene)-hydrazine,
N-(3-methyl-1-phenyl-1H-pyrazolo[3,4-b]pyridin-4-yl)-N′-(2-trifluoromethyl-benzylidene)-hydrazine,
N-(3-methyl-1-phenyl-1H-pyrazolo[3,4-b]pyridin-4-yl)-N′-(6-nitro-benzo[1,3]dioxol-5-ylmethylene)-hydrazine,
4-(3-chloro-4-methoxy-benzylamino)-1-ethyl-1H-pyrazolo[3,4-h]pyridine-5-carboxylic acid
4-(3-chloro-4-methoxy-benzylamino)-1-ethyl-1H-pyrazolo[3,4-h]pyridine-5-(pyridin-4-ylmethyl)-amide,
4-(3-chloro-4-methoxy-benzylamino)-1-ethyl-1H-pyrazolo[3,4-b]pyridine-5-(tetrahydro-furan-2-ylmethyl)-amide,
4-(3-chloro-4-methoxy-benzylamino)-1-ethyl-1H-pyrazolo[3,4-b]pyridine-5-(5-hydroxy-pentyl)-amide,
4-(3-chloro-4-methoxy-benzylamino)-1-ethyl-1H-pyrazolo[3,4-b]pyridine-5-[3-(2-oxo-pyrrolidin-1-yl)-propyl]-amide,
4-tert-butylamino-1-(2-chloro-2-phenyl-ethyl)-1H-pyrazolo[3,4-b]pyridine-5-carboxylic acid ethyl ester,
1-(2-chloro-2-phenyl-ethyl)-4-cyclopropylamino-1H-pyrazolo[3,4-b]pyridine-5-carboxylic acid ethyl ester,
1-(2-chloro-2-phenyl-ethyl)-4-propylamino-1H-pyrazolo[3,4-b]pyridine-5-carboxylic acid ethyl ester,
1-(2-chloro-2-phenyl-ethyl)-4-phenylamino-1H-pyrazolo[3,4-b]pyridine-5-carboxylic acid ethyl ester,
4-butylamino-1-(2-chloro-2-phenyl-ethyl)-1H-pyrazolo[3,4-b]pyridine-5-carboxylic acid ethyl ester,
1-(2-chloro-2-phenyl-ethyl)-4-(2-ethoxy-ethylamino)-1H-pyrazolo[3,4-b]pyridine-5-carboxylic acid ethyl ester,
4-benzylamino-1-(2-chloro-2-phenyl-ethyl)-1H-pyrazolo[3,4-b]pyridine-5-carboxylic acid ethyl ester,
1-(2-chloro-2-phenyl-ethyl)-4-phenethylamino-1H-pyrazolo[3,4-b]pyridine-5-carboxylic acid ethyl ester.

These compounds may be in the form of salt, ester, racemate, active isomer, etc. In one particular embodiment, the neturoprotective compound is chosen from among etazolate, tracazol ate or cartazolate, more preferentially etazolate.

The GABA(A) modulator may be any chemical compound of natural or synthetic origin, notably an organic or inorganic molecule, of plant, bacterial, viral, animal, eukaryote, synthetic or semisynthetic origin, capable of modulating the expression or activity of free radicals (ROS). By way of particular example, benzodiazepines can notably be named.

The compounds or treatments used within the scope of the present invention may be formulated and administered in different manners. Administration may be done by any method known to the person skilled in the art, preferably orally or by systemic or local injection. Injection is typically intraocular, intraperitoneal, intracerebral, intravenous, intra-arterial, subcutaneous or intramuscular. Oral or systemic administration is preferred. The doses administered may be adjusted by the person skilled in the art. Typically, approximately 0.01 mg to 100 mg/kg are injected, for chemical compounds. Particular unit dosages are, for example, from 0.5 to 40 mg per dose administered. It is understood that repeated injections may be performed, possibly in combination with other active agents or any pharmaceutically-acceptable excipient (e.g., buffers, saline or isotonic solutions, in the presence of stabilizers, etc.).

The pharmaceutically-acceptable carrier or excipient may be chosen from among buffer solutes, solvents, binders, stabilizers, emulsifiers, etc. Buffer or diluent solutes are notably calcium phosphate, calcium sulfate, lactose, cellulose, kaolin, mannitol, sodium chloride, starch, powdered sugar and hydroxypropylmethylcellulose (HPMC) (for extended release). Binders are, for example, starch, gelatin, and filler solutes such as sucrose, glucose, dextrose, lactose, etc. Natural or synthetic gums may also be used, such as, notably, alginate, carboxymethylcellulose, methylcellulose, polyvinylpyrrolidone, etc. Other excipients are, for example, cellulose and magnesium stearates. Stabilizers may be incorporated into the formulations, such as, for example, polysaccharides (acacia, agar, alginic acid, guar gum and tragacanch, chitill or its derivatives and cellulose ethers). Solvents or solutes are, for example, Ringer's solution, water, distilled water, phosphate buffers, phosphated saline solutions, and other conventional fluids.

### Sample

To implement the method described above, it is possible to use any sample containing platelets, originating from the treated subject. In fact, the invention shows that neuroprotective compounds are capable of inducing sAPPalpha production in platelets. Thus, the effectiveness of the treatment may be evaluated and monitored by sAPPalpha assay in any sample containing platelets.

In one particular embodiment, the biological sample is a sample of blood or derived from blood. By blood “derived” sample is understood any treated blood sample, for example, by dilution, filtration, purification, etc., in order, for example, to enrich the sample in platelets, eliminate other cell populations, inactivate possible pathogens, calibrate an assay, etc.

The method above can be applied to all mammals, preferably to humans, in particular suffering from neurodegenerative diseases, such as Alzheimer's disease, Parkinson's disease, ALS, Huntington's disease, etc.

sAPPalpha Assay

Different techniques known per se to the person skilled in the art may be used to assay sAPPalpha. Thus, notably, immunological techniques may be mentioned, based on the use of antibodies specific for sAPPalpha. Such antibodies are available in the literature (Exp. Neurol. 2003 September; 183(1):74-80), or can be produced by techniques known in themselves to the person skilled in the art. Thus, it is possible to produce such antibodies by immunization of non-human mammals with sAPPalpha or any epitope or fragment of sAPPalpha, then isolation and/or selection of polyclonal or monoclonal antibodies that can bind sAPPalpha in vitro. The specificity of the antibodies may then be confirmed by determination of binding tests of the antibody to the whole APP protein and/or to other peptides derived from APP protein, such as fragment C83, AICD peptide and P3 peptide. Preferably, antibodies are used that are capable of specifically binding sAPPalpha and incapable of specifically binding the C83 fragment, AICD peptide and P3 peptide. The “specificity” of the binding indicates that binding to sAPPalpha can be discriminated from possible binding to other proteins or peptides.

The method for measuring the production of sAPPalpha can involve an ELISA or RIA technique, the use of substrates coated with specific antibodies, magnetic balls, columns, several antibodies (capture antibodies and detection antibodies), etc. Preferably, an ELISA test is used.

Typically, the production of sAPPalpha measured is compared to a reference level or a value measured before treatment, or at an earlier treatment stage, in said mammal. Thus, it is possible to determine if the level of production sAPPalpha has evolved in the patient consecutive to the treatment or during the treatment. A maintenance or increase of the sAPPalpha level constitutes an indication of the effectiveness of the treatment.

Moreover, the inventors have developed an improved process for immunological dosing of sAPPalpha applicable to any sample. The method relies notably on a sample treatment step, permitting unmasking (and thus making accessible) specific epitopes of the soluble sAPPalpha fragment. In fact, the results presented by the inventors show that, without a suitable protocol, sAPPalpha can not be detected in a specific and quantifiable manner by ELISA.

Thus, another object of the invention resides in a process for immunological dosage of sAPPalpha in a sample, comprising a step of thermal treatment of the sample (to unmask the sAPPalpha epitopes) and a step of immunological dosage. The process is suited to the dosage of sAPPalpha from any sample, and notably blood or blood-derivative samples (serum, platelets, etc), other biological fluids, or culture supernatants. The sample may be pretreated, notably by dilution, enrichment, filtration, etc.

Preferably, the thermal treatment step comprises a treatment of the sample at a temperature comprised between approximately 60° C. and 70° C., during a time period sufficient to unmask the sAPPalpha epitopes, typically for a time period comprised between 30 seconds and 10 minutes, approximately. As shown by the examples, such a method permits reliable, reproducible and specific assay of sAPPalpha from human blood samples.

Immunological assay can be performed by different techniques known per se, such as, notably, ELISA, with any reagent specific for sAPPalpha, notably any specific antibody such as described above. Among these antibodies, any antibody recognizing an epitope contained in amino acid residues 1-17 of APP can notably be named. More specifically, such antibodies or kits are available commercially, such as the ELISA APP kit, sold by Sigma or Biosource, or certain sAPPα specific antibodies (at the level of the APP cleavage) or recognizing sAPPα and APP:
monoclonal antibody 6E10 (specific for sAPPα)
monoclonal antibody 2B3 (included in the IBL sAPPox kit), specific for sAPPα
   monoclonal antibody BAN50, produced by immunization against Abeta peptide 1-16 (PMID): 10480887)
monoclonal antibody 22C11 (anti-APP recognizing sAPPα)
   Polyclonal polyC11 (Upstate/Millipore, Cat # AB5368, produced by Chemicon)
sAPP (poly) from OYC (Cat# APP-KPI-Antiserum)
sAPPα (poly) from Signet Covance (Cat# SIG-39139)
rabbit anti-sAPP antibody 3329, which specifically recognizes the recombinant form of sAPPα (PMID: 9465092).

### Therapeutic Applications

The unexpected proof that the neuroprotective treatments defined above permit inducing or stimulating production of sAPPalpha in platelets permits envisioning new therapeutic uses of this type of compounds.

Thus, an object of the invention resides in the use of a compound chosen from among pyrazolopyridines and GABA (A) receptor modulators for the preparation of a medicament to stimulate or induce sAPPalpha production by platelets in mammals.

The invention also concerns the use of a compound chosen from among pyrazolopyridines and GABA (A) receptor modulators for the preparation of a medicament to reduce the risk of thrombus formation in mammals.

The invention also concerns the use of a compound chosen from among pyrazolopyridines and GABA (A) receptor modulators for the preparation of a medicament for reducing vascular complications in patients suffering from neurodegenerative diseases.

The invention also concerns the use of a compound chosen from among pyrazolopyridines and GABA (A) receptor modulators for the preparation of a medicament for inhibiting platelet aggregation in mammals, in particular in patients suffering from neurodegenerative diseases.

The present invention will be described in more detail by means of the examples that follow, which may be considered as illustrative and non-limiting.

### FIGURE LEGEND

FIG. 1: sAPPalpha assay in non-treated serum

FIG. 2: Effect of a thermal treatment on the detection of sAPPalpha by ELISA

FIG. 3: Detection of sAPPalpha in human serum

FIG. 4: Detection of recombinant sAPPalpha in serum

FIG. 5: Etazolate stimulates the in vitro production of sAPPalpha

FIG. 6: Etazolate stimulates the production of sAPPalpha in neurons

FIG. 7: Etazolate stimulates the in vivo production of sAPPalpha

FIG. 8: Effect of etazolate on the toxicity of the amyloid peptide and the effect of GABA_{A} inhibitors on the neuroprotection induced by etazolate. Statistics: Wilcoxon Test: ###, p<0.001; **, p<0.0; ***, p<0.001

FIG. 9: Inhibitor effect of alpha secretase on the neuroprotection induced by etazolate. Statistics: Wilcoxon Test: *, p<0.05, ***, p<0.001

FIG. 10: Effect of an anti-sAPPα neutralizing antibody on the neuroprotection induced by etazolate. Statistics: Wilcoxon Test: ##, p<0.01, ***, p<0.001

### EXAMPLES

### Example 1

### Process for sAPP Alpha Assay

The soluble fragment of APP (sAPPα) circulating in the blood comes from platelet cells and the associated α-secretase activity. This fragment was shown to decrease with age and during the physiopathological process of Alzheimer's disease (AD). The sAPPα circulating in the blood may be considered a biomarker for monitoring changes in APP processing that appear with age and during the physiopathological process of Alzheimer's disease and that may be corrected by taking medicinal treatments.

Therefore, there is a real interest in precisely quantifying sAPPα levels in the blood, and more particularly in the serum, after blood clotting and platelet activation, in order to evaluate the effectiveness of treatments whose purpose is to modify APP processing for treating Alzheimer's disease.

The method described below was developed in order to unmask and render accessible the specific epitope of the sAPPα soluble fragment for high affinity antibody-antigen detection according to the double sandwich ELISA technique.

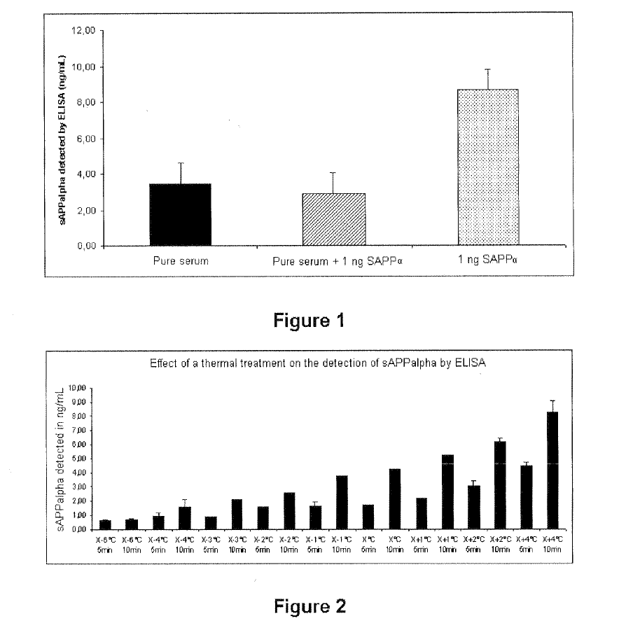

Indeed, without a suitable protocol for treating serum samples, sAPPα could not be detected in a quantifiable and specific manner by ELISA. As FIG. 1 shows, serum alone without pretreatment shows a very clearly quantifiable detection of sAPPα (3.5 ng/mL), but it does not seem to increase with added recombinant sAPPα (+10 ng/mL), while the same quantity of sAPPα added shows a detection (8.7 ng/mL) at about the expected quantity (10 ng/mL). ELISA detection in pure serum without particular treatment does not seem to be specific to soluble and circulating sAPPα.

In order to have access to soluble sAPPα in the serum and be able to quantify it in a reliable, specific and reproducible manner, we have developed a sample preparation and treatment method that permits rendering the sAPPα present in the serum accessible to the specific ELISA antibodies by a thermal treatment.

The serum samples are initially diluted in pH 7.4 Dulbecco's phosphate buffered saline (PBS) (Sigma # D8537), 5% BSA, 0.05% Tween-20. The diluted samples are then heat-treated at 66° C. for 10 minutes, and then cooled at 4° C. The samples are then analyzed by the ELISA technique by means of a kit specific for sAPPα.

As FIG. 2 shows, the detection of sAPPα by ELISA from serum increases in a significant manner as a function of the heating temperature and the thermal treatment duration (temperature range 60-70° C.; X=66° C.).

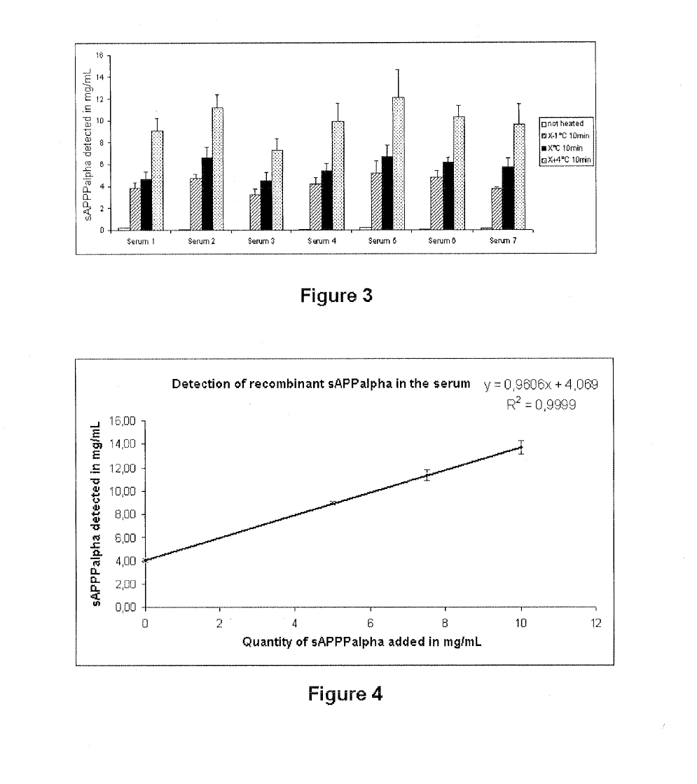

This serum sample preparation and thermal treatment method was evaluated in 7 different human serums, in 3 separate experiments. As FIG. 3 shows, the detection of sAPPα by ELISA from human serums appears to be reproducible with this method (X=66° C.).

In order to better evaluate this method for sAPPα, in serum, we evaluated the linearity of sAPPα detection in a human serum by using three increasing quantities of sAPPα (5, 7.5 and 10 ng/mL). FIG. 4 shows the mean of 3 separate experiments conducted on the same serum on 3 different days.

As FIG. 4 shows, there is a very good proportionality relationship between the added/spiked sAPPα quantities and the sAPPα quantities detected by ELISA in the biological matrix (serum) after preparation and thermal treatment. These results show that sAPPα added to the treated serum does not compete with the free sAPPα of the serum. These results show good reproducibility over 3 experiments conducted on 3 different days.

From these results, the recovery of recombinant sAPPα in the treated serum could be calculated in comparison with samples corresponding to the biological matrix without endogenous sAPPα and to which the same quantities of sAPPα were added.

As the table below shows, the recovery (expressed in % of the expected value) of recombinant sAPPα in the serum at 3 increasing quantities is situated within acceptable limits 100%±25%.

Recovery of recombinant sAPPα in the serum

| | expected concentrations (ng/mL) | | |
|---|---|---|---|
| Samples | 5 | 7.5 | 10 |
| 1/aliquot-1 | 106.0 | 95.2 | 100.7 |
| 1/aliquot-2 | 122.2 | 113.1 | 114.2 |
| 1/aliquot-3 | 118.9 | 103.5 | 101.1 |
| 1/aliquot-4 | 113.4 | 109.9 | 111.3 |
| 1/aliquot-5 | 119.2 | 104.1 | 101.2 |
| Recovery mean | 115.9 | 105.2 | 105.7 |
| Standard deviation | 6.4 | 6.9 | 6.5 |
| CV % | 5.5 | 6.5 | 6.2 |

The FDA defines the performance criteria for ELISA assays applied to diagnostic processes in the document US Food And Drug Administration Guidance for Industry, Bioanalytical Method Validation, May 2001. The following documents specify the acceptance and validation criteria for immunoassays.
Findlay et al. Validation of immunoassays for bioanalysis: a pharmaceutical industry perspective. Journal of Pharmaceutical and Biomedical Analysis 21 (2000) 1249-1273
Viswanathan et al. Workshop/Conference Report—Quantitative Bioanalytical Methods Validation and Implementation: Best Practices for Chromatographic and Ligand Binding Assays. The AAPS Journal 2007; 9 (1) Article 4 (http://www.aapsj.org)

The data below show that the performance of the assay method of the invention, implemented by means of the sAPPα kit sold by IBL, complies with the criteria recommended by the FDA.

| | | Concentration (ng/ml) | | |
|---|---|---|---|---|
| | sAPPα | 5 | 7.5 | 10 |
| Recovery | % Recovery | 115.9 | 105.2 | 105.7 |
| | CV % | 5.5 | 6.5 | 6.2 |
| Inter-series | CV % | 4.6 | 6.4 | 5.5 |
| precision | | | | |
| Intra-series | CV % | 5.3 | 5.3 | 1.9 |
| precision | | | | |
| Linearity | r2 | 0.99923 | | |
| | CV % | 1.8 | 4.0 | 4.1 |
| | Accuracy % | 98.1 | 96.6 | 96.0 |
| LOQ (ng/ml) | | 1.0 | | |
| Matrix effect | % Recovery | 95.8 | | |
| | CV % | 13.1 | | |
| | Accuracy % | 90.0 | | |
| Specificity | % | 95.1 | | |
| | CV % | 6.2 | | |

LOQ: limit of quantification; CV % coefficient of variation

### Example 2

### Etazolate Stimulates the Production of sAPPalpha

HEK293 cells transfected in a stable manner over-expressing human APP were kept in Modified Eagle medium containing Earle salt and supplemented by 10% fetal calf serum (FCS), 2 mM L-glutamine (Sigma, Lyon, France), 1× Nonessential Amino Acids and antibiotics. The cells were treated for 48 hours after spreading variable concentrations of the molecules indicated on 10-cm plates, or with DMSO as a carrier, for 24 hours. The sAPPalpha had been measured by ELISA and Western blot by means of commercially available antibodies.

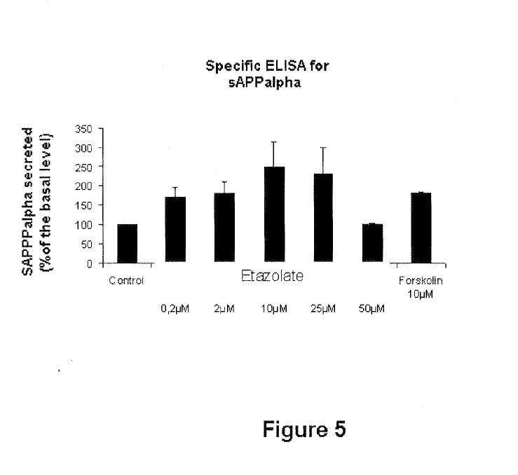

The results obtained are presented in FIG. 5 and show that etazolate induces sAPPalpha secretion.

### Example 3

### Etazolate Stimulates the Production of sAPPalpha by Cortical Neurons

The production of sAPPalpha was measured in cortical neurons isolated from 17-day-old Wistar rat embryos. The cells are obtained from cortical structures that are dissected into a solution containing 0.25% trypsin. The dissociated cells are seeded at a density of 500,000 per cm² in a Neurobasal medium containing additives (1×B27, 2 mM L-glutamine, 0.6% glucose, antibiotics and antimycotics, as well as 2% horse serum) in culture dishes coated with 6 μg/mL, of polyornithine. The cells are kept at 37° C. and 5% CO₂. 24 hours after seeding, the cells are treated with 5 μM AraC (5 cytosine arabinofuranoside) as an antimitotic agent. After 4 days in vitro, half the medium is exchanged for medium without horse serum and the culture is kept for maturation in this medium for 7 to 10 days.

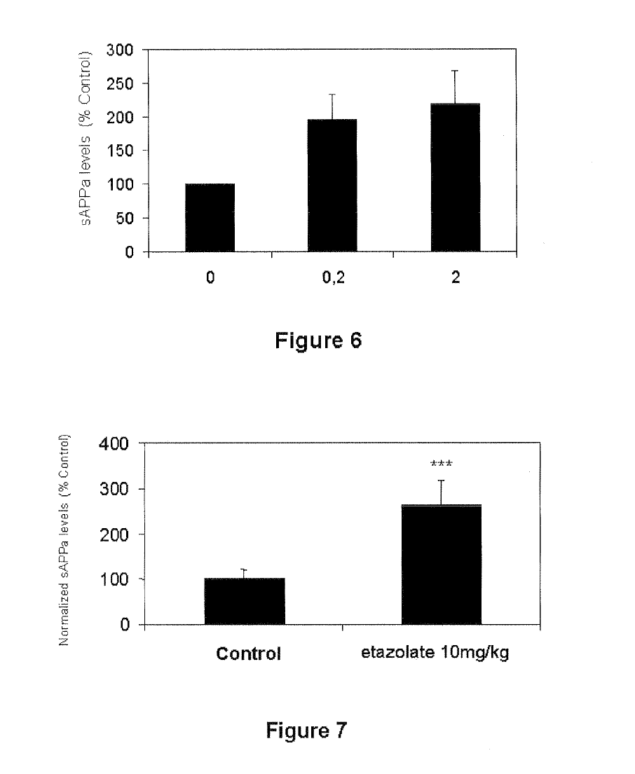

The sAPPalpha was measured by Western blot by means of antibodies available commercially after one change of medium and accumulation in fresh medium for 24 hours. Quantification was done by densitometric analyses of scanned autoradiographic images. As FIG. 6 shows, etazolate (0.2 and 2 μM for 24 h) stimulates the salting out of sAPPalpha from cortical neurons. The results presented are the mean±SEM of three independent experiments performed in duplicate and are expressed the form of percentage of the control (non-treated cultures).

### Example 4

### Etazolate Stimulates the Production of sAPPalpha In Vivo

The production of sAPPalpha was studied in vivo in the guinea pig, a physiological model for APP processing in the brain. The etazolate or the excipient (physiological saline solution) was administered to male Hartley albino guinea pigs, weighing 250-270 g at the beginning of the experiment, once a day for 15 consecutive days, per os in a dose of 10 mg/kg. One h after the last administration, the guinea pigs were sacrificed and the brains immediately extracted, frozen in nitrogen and stored at −80° C. The cortices were homogenized at 4° C. in a pH 7.5 20 mM Tris base solution containing 0.2% Triton X-100, 50 μg/mL, gentamicin and a protease inhibitor cocktail. The soluble sAPPalpha was measured by an ELISA test and the results normalized with regard to the quantity of proteins present in the extracts.

FIG. 7 shows the increase of the quantity of sAPPalpha measured in the brains of animals treated with etazolate, compared to control animals treated with the excipient. The increase by a factor of three induced by etazolate is statistically very significant (***: p<1E-4 according to the Wilcoxon test).

The results obtained show that etazolate induces sAPPalpha secretion.

### Example 5

### The Neuroprotective Effect of Etazolate Requires the Production of sAPPalpha In Vivo

Aβ25-35 peptide contains the neurotoxic fragment of the amyloid peptide and is a tool classically used to study the neuroprotective effects of compounds. At the beginning of each experiment, the neuronal cultures aged 7-10 days are changed with fresh culture medium and treated with the etazolate inhibitor compound, six hours before the addition of Aβ25-35 amyloid peptide at a concentration of 33.5 μM In a classical and reproducible manner, this concentration generates 30% to 40% toxicity in neuronal cultures.

Several experiments are undertaken to characterize the neuroprotective effect of etazolate. In order to verify whether the neuroprotection involves the GABAA receptor, GABAA receptor antagonists Picrotoxine (PTX), Gabazine/SR95531, and Bicuculine (BIC) are pre-incubated one hour before the etazolate at a concentration of 50 μM, 20 μM and 10 μM, respectively.

Recently, several studies have shown that sAPPα has neurotrophic and neuroprotective functions, notably against amyloid peptide in vitro and in vivo, suggesting that the etazolate could mediate its neuroprotective effects via the alpha secretase pathway. In order to determine whether it is the inhibition of sAPPalpha or its production that provides the neuroprotective effect of etazolate against amyloid peptide, a neutralizing anti-sAPPα antibody (3E9 antibody) and alpha secretase inhibitors are respectively used. For the neutralization of sAPPα, 3E9 antibody (5 μg/ml) is added to the cortical cells at the same time as the etazolate. Two alpha secretase inhibitors, the compound Furin Inhibitor I (Hwang E M, Kim S K, Sohn J H, Lee J Y, Kim Y, Kim Y S, Mook-Jung I. Furin is an endogenous regulator of alpha-secretase associated APP processing. Biochem Biophys Res Commun. 2006 Oct. 20; 349(2):654-9.) and TAPI (Slack B E, Ma L K, Seah C C. Constitutive shedding of the amyloid precursor protein ectodomain is up-regulated by tumour necrosis factor-alpha converting enzyme. Biochem J. 2001 Aug. 1; 357(Pt 3):787-94) are used in pretreatment one hour before the addition of the etazolate.

All the treatments are effected at least twice and in at least two different cultures. After an incubation of 48 hours, the toxicity is measured by an MTT test. The results, normalized to the untreated mean, are statistically analyzed by the Wilcoxon test. The significant value is determined at p less than or equal to 0.05.

### MTT:

The toxicity is measured by using the MTT test. After incubation with the compounds, MTT is added at a final concentration of 0.5 mg/mL by wells. The plates are then incubated for 30 minutes at 37° C. at night. The medium is drawn off and the crystals are resuspended in 500 μL of DMSO (dimethylsulfoxide). The absorbance at 550 nm is read and the viability percentage is calculated.

### Results:

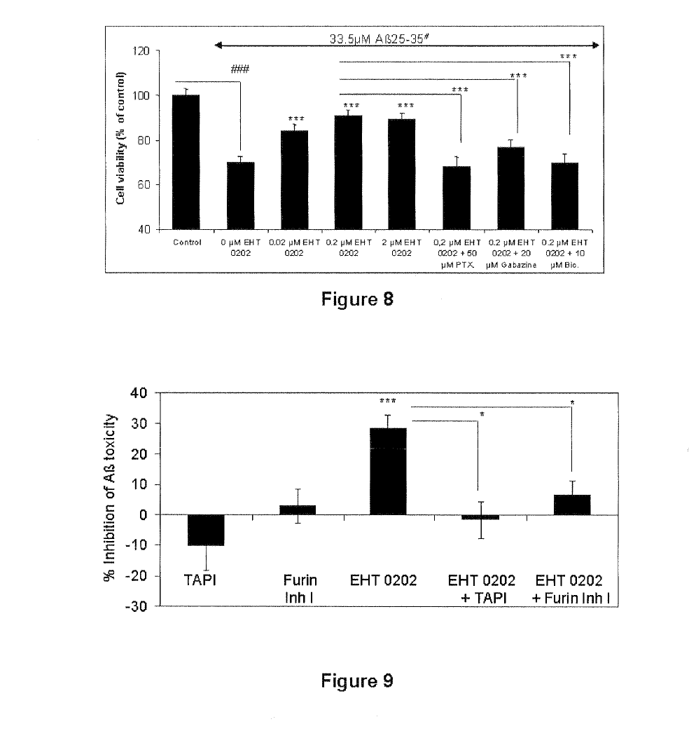

The results obtained are shown in FIGS. 8-10. These results illustrate the protective effect of the compound of the invention on neuronal death induced by amyloid peptide Aβ 25-35.

During the co-treatment of neurons by etazolate, a dose-dependent protector effect is observed (FIG. 8) with, in particular, 90% cellular viability obtained for the dose of 0.2 μM. This effect is blocked by the use of the three GABA_{A} inhibitor agents and statistical analysis indicates that this effect is highly significant (p<1e-4 with the Wilcoxon test after comparison 0.2 μM EHT 0202 versus 0.2 μM EHT 0202 more antagonists). The results correspond to the means±SEM of the seven independent experiments.

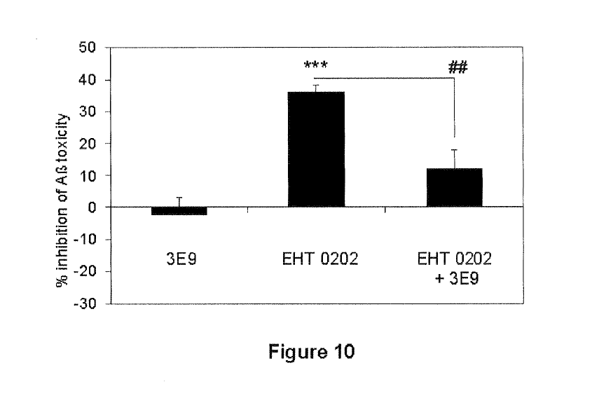

FIGS. 9 and 10 show the results obtained by means of etazolate on cortical neurons in the presence of inhibitors of the production or activity of sAPPα. The results presented show that etazolate permits attaining a protective effect on these cells that is inhibited by treatment with two alpha secretase inhibitors, the compound Furin Inhibitor I and TAPI (FIG. 9). These data indicate that the activity of alpha secretase responsible for the production of sAPPα is necessary to the neuroprotection induced by etazolate.

FIG. 10 shows that the neuroprotection induced by etazolate requires the production of sAPPα, since the neuroprotective effect of etazolate is lost when an anti-sAPPα neutralizing antibody is added to the culture medium.

The present invention documents the neuroprotective effect of etazolate on the toxicity induced by amyloid peptide as acting via the GABA_{A} receptor. This neuroprotective effect is associated with the activation of the alpha secretase pathway and the production of sAPPα.

1. A process for immunological dosage of sAPPalpha in a sample, comprising a step of thermal treatment of the sample and a step of immunological dosing.

2. Process according to claim 1, characterized in that the sample is a sample of blood or derived from blood or other biological fluids.

3. Process according to claim 1, characterized in that the thermal treatment step comprises a treatment of the sample at a temperature comprised between approximately 60° C. and 70° C., during a time period sufficient to unmask sAPPalpha epitopes, typically for a time period comprised between 30 seconds and 10 minutes, approximately.

4. Process according to claim 1, characterized in that the immunological dosage step is performed by means of a specific antibody.

5. Use of a process according to claim 1 for the dosage of sAPPalpha in a sample (derivative) of human blood.

6. Use according to claim 5, for the dosage of sAPPalpha in a blood (derivative) sample originating from a human subject with Alzheimer's disease.

7. Use of a process according to claim 1 to evaluate the efficacy of a treatment in a human subject with Alzheimer's disease.

8. A method to evaluate or monitor the effectiveness of a neuroprotective treatment in mammal, comprising a step of measuring in vitro or ex vivo the production of sAPPalpha in a biological sample from the mammal having received said treatment, said sample containing platelets, the production of sAPPalpha being an indication of treatment effectiveness.

9. Method according to claim 8, characterized in that the neuroprotective treatment is a compound chosen from among pyrazolopyridines and GABA (A) receptor modulators.

10. Method according to claim 8, characterized in that the mammal has a neurodegenerative disease.

11. Method according to claim 8, characterized in that the biological sample is a blood or blood derivative sample.

12. Method according to claim 8, characterized in that the production of sAPPalpha is measured by an immunological test, preferably ELISA.

13. Method according to claim 8, characterized in that the production of sAPPalpha measured is compared to a reference level or to a value measured before treatment, or at an earlier treatment stage, in said mammal.

14. Use of a compound chosen from among pyrazolopyridines and GABA (A) receptor modulators for the preparation of a medicament to stimulate or induce sAPPalpha production by platelets in a mammal.

15. Use of a compound chosen from among pyrazolopyridines and GABA (A) receptor modulators for the preparation of a medicament to reduce the risk of thrombus formation in a mammal.

16. Use of a compound chosen from among pyrazolopyridines and GABA (A) receptor modulators for the preparation of a medicament for reducing vascular complications in patients with neurodegenerative diseases.

17. Use of a compound chosen from among pyrazolopyridines and GABA (A) receptor modulators for the preparation of a medicament for inhibiting platelet aggregation in a mammal, in particular in patients with neurodegenerative diseases.
